(19) United States
(12) Patent Application Publication
(10) Pub. No.: US 2026/0083204 A1
(43) Pub. Date: Mar. 26, 2026

(54) CLIP DEVICE FOR STABILIZING ITEMS

(71) Applicant: Gregory A. DUARTE, Los Angeles, CA (US)

(72) Inventor: Gregory A. DUARTE, Los Angeles, CA (US)

(21) Appl. No.: 19/340,802

(22) Filed: Sep. 25, 2025

Related U.S. Application Data
(60) Provisional application No. 63/699,141, filed on Sep. 25, 2024.

(51) Int. Cl. A42B 3/14; A42B 3/32; A61F 9/02

(52) U.S. Cl. CPC A42B 3/142; A42B 3/324; A61F 9/027; A61F 9/029

(57) ABSTRACT

A clip device that secures items together to stabilize movement. An apparatus including the clip device and a strap is secured to a portion of a helmet, ideally so that the strap extends over the top of a wearer's head from ear to ear, to stabilize headwear during use.

### CROSS-REFERENCE TO RELATED APPLICATIONS

This application claims the benefit of U.S. Provisional Application Ser. No. 63/699,141 filed Sep. 25, 2024, incorporated by reference.

### FIELD OF THE INVENTION

The invention relates generally to devices that secure items together. More specifically, the invention relates to a clip device that secures items together to stabilize movement. While the invention is discussed with respect to an apparatus used in the sports industry, it is contemplated that the clip device may be applicable to any industry, including, for example, medical, gaming, construction, packaging, outdoor, drone, and military.

### BACKGROUND OF THE INVENTION

The invention is discussed with respect to an apparatus used with headwear, i.e., that worn to protect the head and/or face, for example, visors, face shields, helmets, goggles, and headwear comprised of both a helmet with goggles, otherwise referred to as a mask. However, it is contemplated that the invention may be used with any item, or with an apparatus used with any item.

Headwear such a helmets, goggles, and masks are worn in many applications. There are many different brands and types of headwear, each with its own unique design features and construction. Universal headwear for use with all applications or activities does not exist.

Headwear is worn during activities such as skiing, snowboarding, ATV riding, motorcycling, military training and combat, drone or augmented reality/virtual reality applications or games, medical operations, welding, to name a few.

Often times, headwear is or becomes unstable in that it moves around or shifts from a proper position on the wearer which may lead to obstructed vision or unprotected parts like eyes or ears. Or during use, headwear wiggles, or vibrates, causing discomfort and irritation. And after prolonged use, headwear can slip to weigh heavily on the wearer's nose and/or ears, or even strain the wearer's head and neck.

What is needed is a device that secures items together and that can be used with an apparatus to stabilize headwear during use. The present invention satisfies this need.

### SUMMARY OF THE INVENTION

The invention is directed to a clip device that secures items together to stabilize movement.

For exemplary purposes, the invention will be described with reference to an apparatus used with headwear worn in the sports industry, and specifically to paintball sports, but again, any sport or activity is contemplated in which protective headwear is worn.

A common and well documented risk of headwear failing occurs during paintball practice or competition, thus exposing a player's face and eyes to fast moving high velocity paintballs which can cause serious injury. Further, headwear that fails during paintball sport exposes the wearer to mud, water, air-soft ammunition, dust, debris, shrapnel, or other potential threats.

The invention is directed to a clip device that secures items together. The clip device comprises a primary clip, and may further include an adapter clip that works in conjunction with the primary clip.

The clip device may further be connected to a strap forming an apparatus used with headwear.

According to an embodiment of the invention, an apparatus comprises a primary clip component connected at both ends of a strap. Each clip component is secured to a portion of a helmet, ideally so that the strap extends over the top of a wearer's head from ear to ear.

According to another embodiment of the invention, an apparatus comprises a primary clip component connected at both ends of a strap, and further an adapter clip component connected to each primary clip component. Each adapter component is secured to a portion of the goggles, specifically the band portion of the googles, and ideally so that the strap extends over the top of a wearer's head from ear to ear.

The invention stabilizes headwear to provide a safe and comfortable experience for sports players. The invention essentially “locks” headwear to a wearer to prevent the headwear from shifting, wiggling, vibrating, or slipping. Thus, the wearer constantly remains protected improving safety by reducing risk of injury. Further, the invention may alleviate weight experienced on the nose and/or ears of a wearer or strain on the wearer's head and neck.

Advantageously, the invention can be used universally with all brands and types of helmets, googles, masks, visors, face shields, etc. An apparatus including clip device may be used with any brand or type of google such as ski googles, bicycle motocross (BMX) googles, or first person view (FPV) goggles, virtual reality (VR) headwear, face shields such as personal protective equipment (PPE), welding helmets, or custom fit high impact polycarbonate masks like those used by basketball players, and masks used in tactical and recreational sports such as paintball or airsoft.

The invention and its attributes and advantages will be further understood and appreciated with reference to the detailed description below of presently contemplated embodiments, taken in conjunction with the accompanying drawings.

### BRIEF DESCRIPTION OF THE DRAWINGS

FIG. 1 is a non-limiting, exemplary perspective view of a primary clip component of a clip device.

FIG. 2 is a non-limiting, exemplary front view of a primary clip component.

FIG. 3 is a non-limiting, exemplary rear view of a primary clip component.

FIG. 4 is a non-limiting, exemplary perspective view of a primary clip component in an open or unlocked state.

FIG. 5 is a non-limiting, exemplary side view of a primary clip component in an open or unlocked state.

FIG. 6 is a non-limiting, exemplary perspective view of a primary clip component in a closed or locked state.

FIG. 7 is a non-limiting, exemplary side view of a primary clip component in a closed or locked state.

FIG. 8 is a non-limiting, exemplary view of an apparatus comprising a primary clip component.

FIG. 9 is a non-limiting, exemplary view of the apparatus attached to a helmet.

FIG. 10 is a non-limiting, exemplary view of the apparatus attached to another helmet.

FIG. 11 is a non-limiting, exemplary close up view of the attached apparatus.

FIG. 12 is a non-limiting, exemplary perspective view of an adapter component of a clip device.

FIG. 13 is a non-limiting, exemplary front view of an adapter component.

FIG. 14 is a non-limiting, exemplary rear view of an adapter component.

FIG. 15 is a non-limiting, exemplary side view of an adapter component.

FIG. 16 is a non-limiting, exemplary perspective view of an exploded assembly comprising a primary clip component and an adapter component.

FIG. 17 is a non-limiting, exemplary perspective view of an assembly with a primary clip component and an adapter component.

FIG. 18 is a non-limiting, exemplary view of an apparatus comprising and assembly with a primary clip component and an adapter component.

FIG. 19 is a non-limiting, exemplary view of the apparatus attached to goggles.

### DETAILED DESCRIPTION OF THE INVENTION

The invention relates generally to devices that secure items together. More specifically, a non-limiting, exemplary device relates to a clip device that secures items together to stabilize movement.

The clip device comprises a primary clip, and may further include an adapter clip that works in conjunction with the primary clip. The clip device may further be connected to a strap forming an apparatus used with headwear.

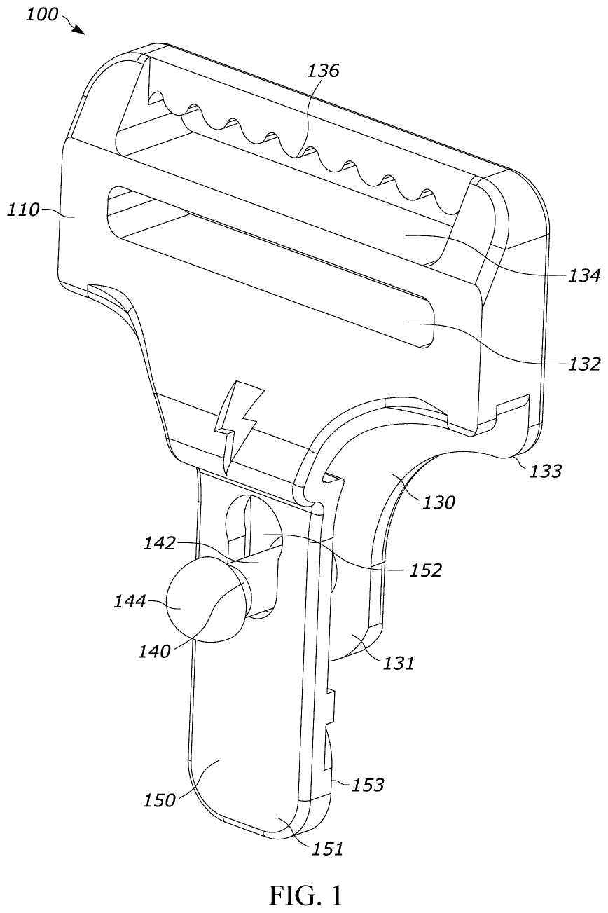

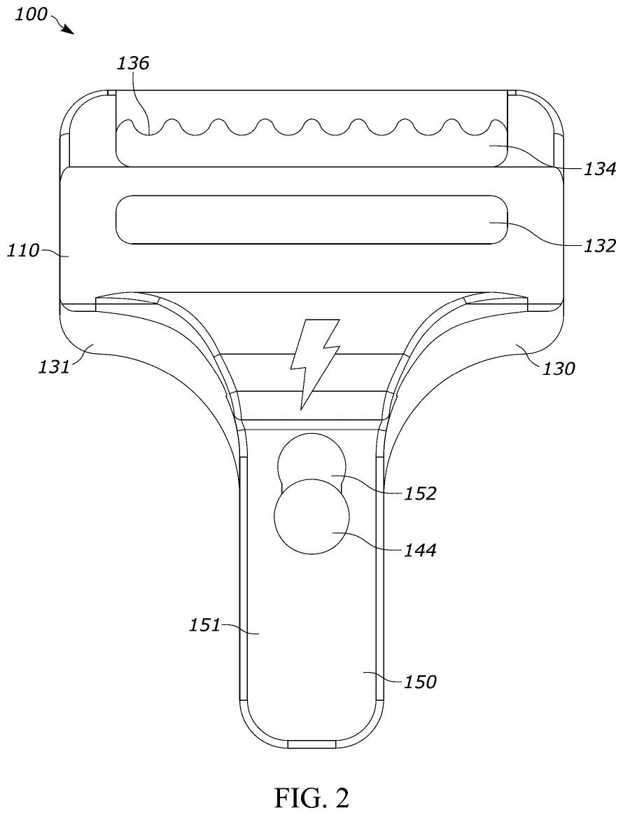

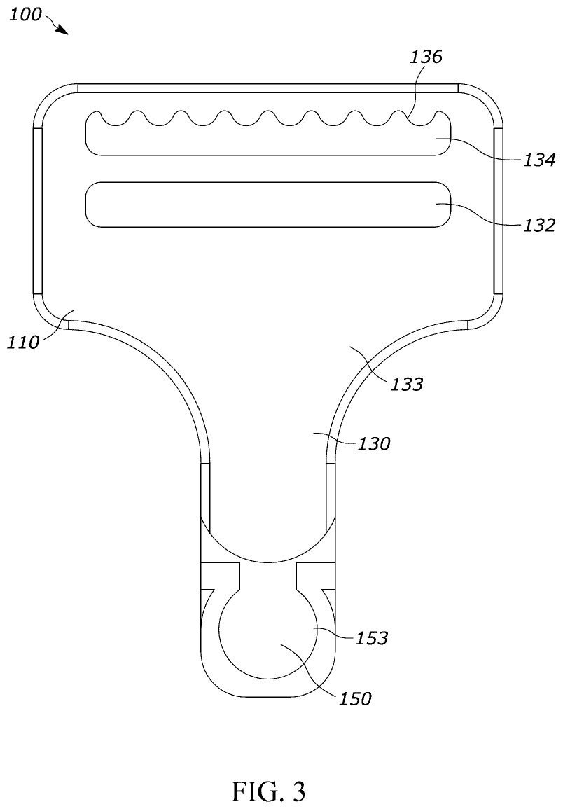

FIG. 1 through FIG. 7 illustrates a clip device 100 comprising a primary clip component 110. The primary clip component 110 includes a base portion 130 and a moveable flap portion 150. The base portion 130 includes a first slit element 132 and a second slit element 134. The second slit element 134 further includes a plurality of spike elements 136.

The base portion 130 includes a front surface 131 and an opposing back surface 133. A protrusion element 140 extends from the front surface 131 and include a shaft portion 142 and a bulb portion 144.

The flap portion 150 includes a top surface 151 and a rear surface 153. The flap portion 150 is hingedly connected to the base portion 130 such that it is moveable with respect to the base portion 130. The flap portion 150 includes a slot element 152.

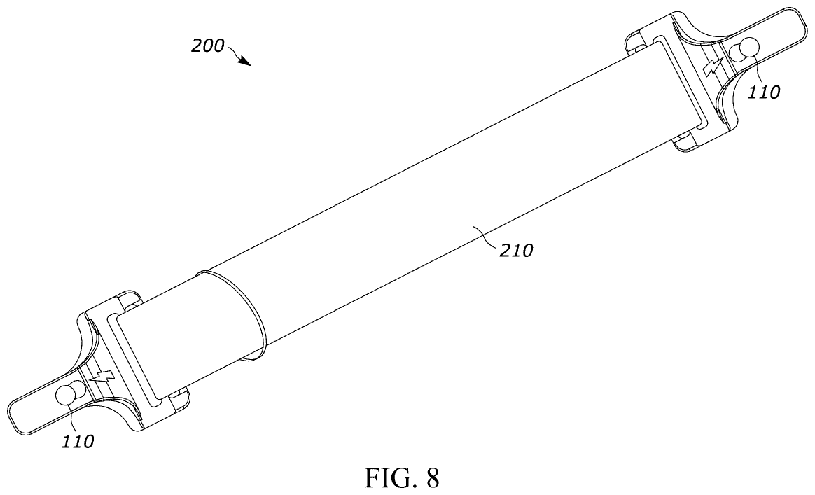

As shown in FIG. 8, an apparatus 200 comprises a primary clip component 110 connected at both ends of a strap 210. Each end of the strap 210 is routed through the first slit element 132 and the second slit element 134 so that the strap 210 is tight with little or no slack. Additionally, the plurality of spike elements 136 are configured to impress upon the material of the strap 210 further preventing movement of the strap 210 within the primary clip component 110.

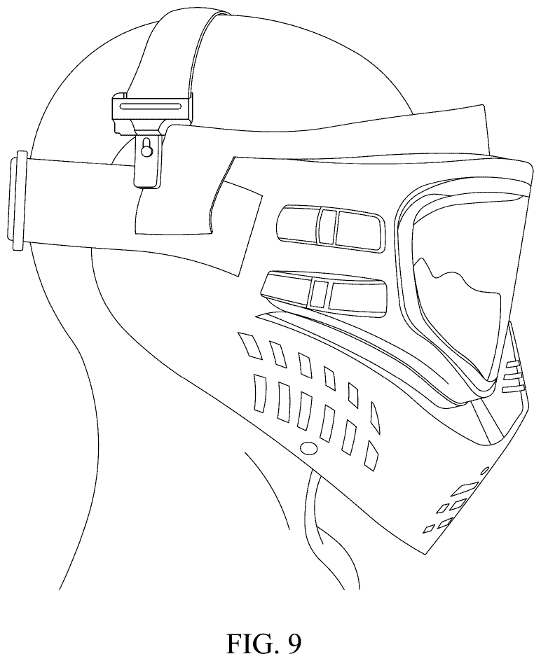

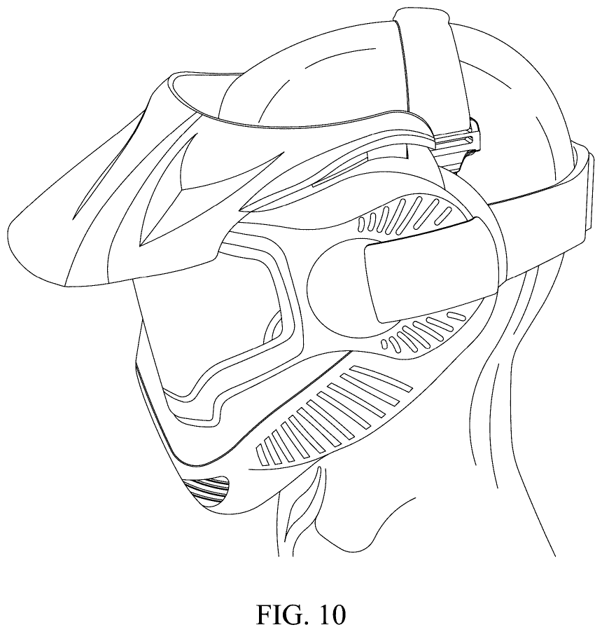

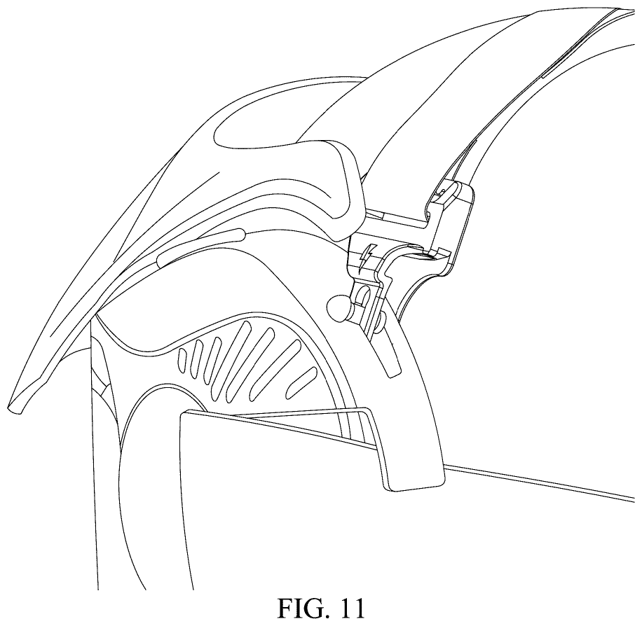

Each primary clip component 110 is secured to a portion of a helmet as shown in FIG. 9 through FIG. 11. For example, the primary clip component 110 is positioned about a vent opening near the ear area of a helmet. The back surface 133 of the base portion 130 is located on the wearer's head with the strap 210 extending over the top of a wearer's head from ear to ear.

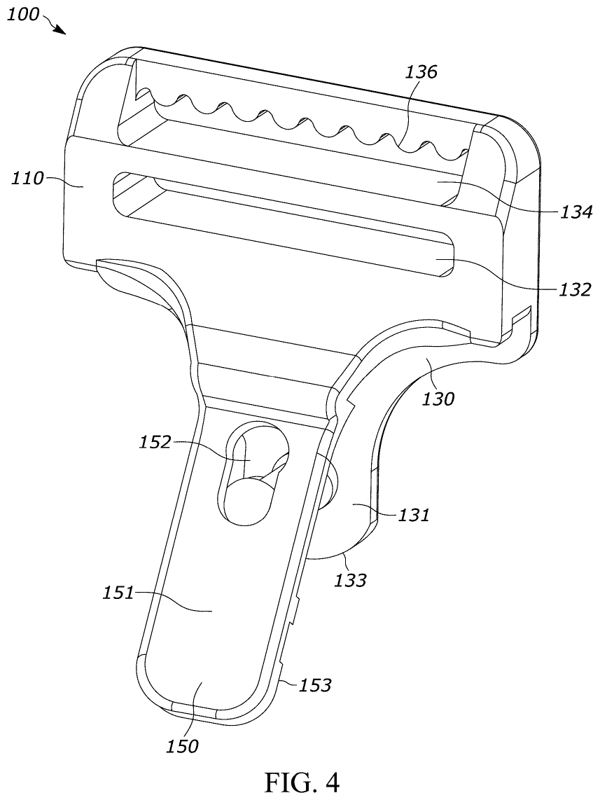

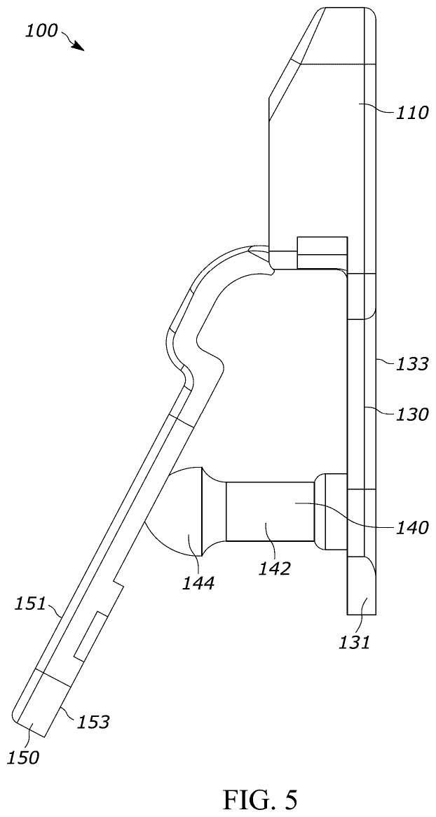

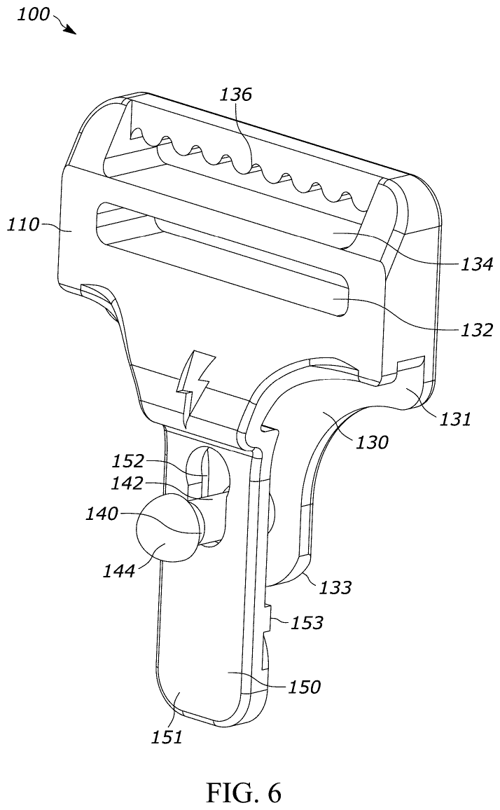

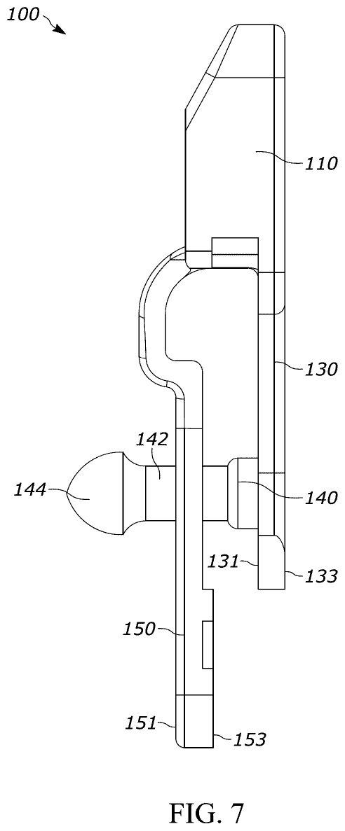

The clip component 510 is positioned about a vent opening and the flap portion 150 moveable to provide an open or unlocked state as shown in FIG. 4 and FIG. 5. The permits clearance for the flap portion 150 to encapsulate a portion of the headwear while in a closed or locked state as shown in FIG. 6 and FIG. 7. In the closed or locked state, the protrusion element 140 is positioned through the slot element 152 of the flap portion 150. The slot element 152 receives the shaft portion 142 such that the bulb portion 144 pierces through both the top surface 151 and the rear surface 153. The bulb portion 144 is larger in diameter than the width of the slot element 152 so that the protrusion element 140 fastens with an interference fit with the primary clip component 110.

As shown in FIG. 4, the slot element 152 may comprise of two sections-a first section with a first opening larger than a second opening of a second section. The protrusion element 140 may be directed to the first section with larger opening, for example by pulling downwardly on the flap portion 150, so that the bulb portion 144 can pass through the slot element 152. The clip component 510 achieving an open or unlocked state as shown in FIG. 4 and FIG. 5.

The strap 210 may be adjustable, for example using a lever or ratchet-type mechanism, or a fixed length. The strap 210 may comprise of any thin material, such as a textile, leather, plastic, nylon, rubber, cloth, elastic, carbon fiber, polyester, to name a few.

The clip device 100 may be constructed from any durable material while also permitting the flap portion 150 to move back and forth. Examples of materials from which the clip device may be constructed include plastic, rubber including urethane rubber, or event metal.

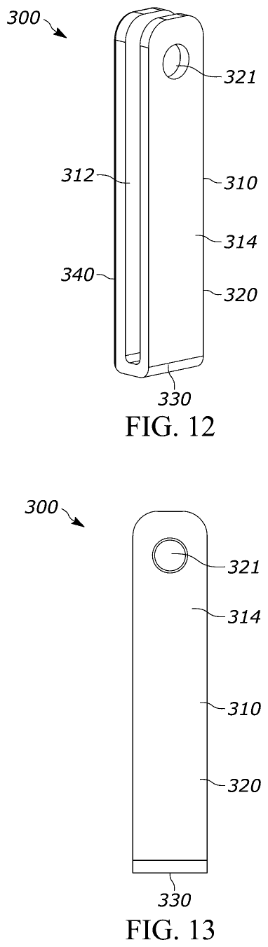

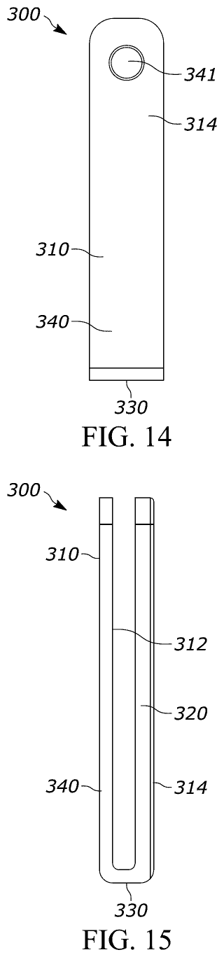

FIG. 12 through FIG. 15 illustrates an adapter clip component 300. The adapter clip component 300 is in the form of a u-shaped body 310 including a first leg element 320 connect through a platform element 330 to a second leg element 340. The body 310 includes an inside surface 312 and outside surface 314. Each leg element 320, 340 includes an aperture element 321, 341.

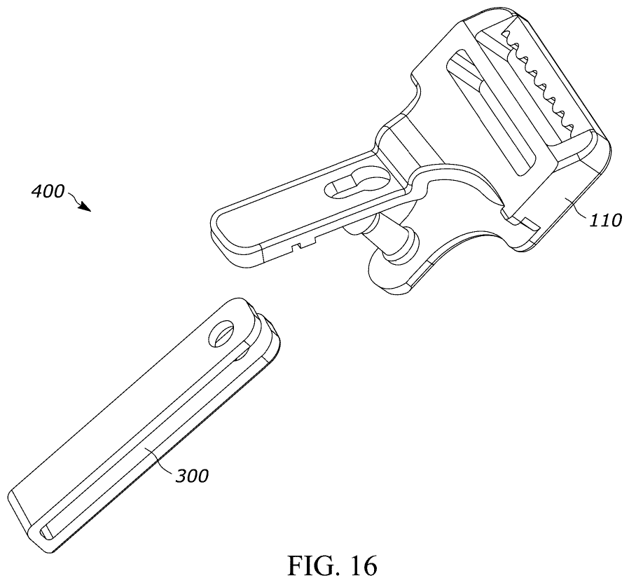

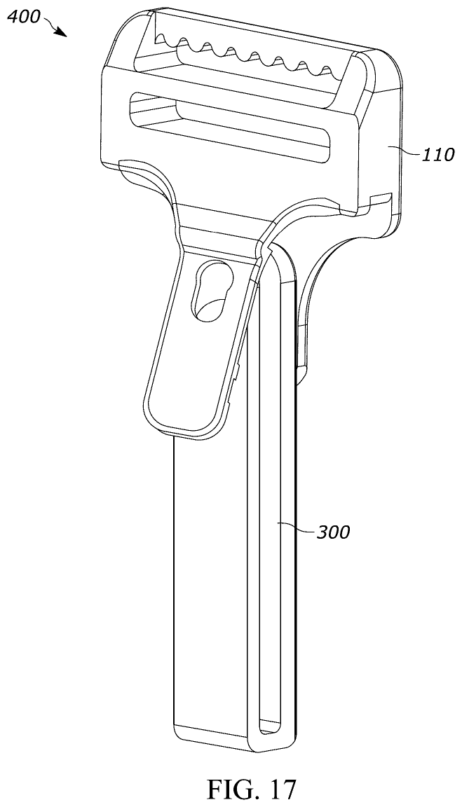

FIG. 16 illustrates a perspective view of an exploded assembly 400 comprising a primary clip component 110 and an adapter component 300. As shown in FIG. 17, the primary clip component 110 and adapter component 300 are assembled. Specifically, the aperture elements 321, 341 are aligned with the protrusion element 140 such that the outside surface 314 of the adapter clip component 300 is positioned on the front surface 131 of the base portion 130 of the primary clip component 110 and the rear surface 153 of the flap portion 150 of the primary clip component 110.

The protrusion element 140 is positioned through the aperture elements 321, 341 of the adapter clip component 300. Specifically, the bulb portion 144 pierces through both the outside surface 314 and inside surface 312 of the body 310 of the adapter clip component 300 as well as the top surface 151 and the rear surface 153 of the flap portion 150 of the primary clip component 110. The bulb portion 144 is smaller in diameter than the width of the aperture elements 321, 341 so that the protrusion element 140 easily passes through the adapter clip component 300 and fastened with an interference fit to the primary clip component 110.

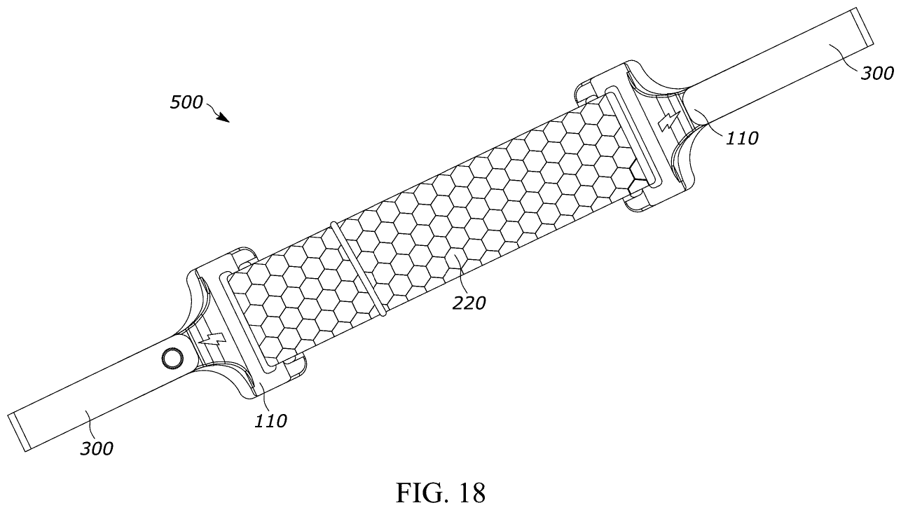

FIG. 18 illustrates an apparatus 500 comprising and assembly 400 with a primary clip component 110 and an adapter component 300. Like that mentioned above, the primary clip component 110 is connected at both ends of a strap 220 and further connected to the adapter clip component 300. Each end of the strap 220 is routed through the first slit element 132 and the second slit element 134 so that the strap 220 is tight with little or no slack. Additionally, the plurality of spike elements 136 are configured to impress upon the material of the strap 220 further preventing movement of the strap 220 within the primary clip component 110.

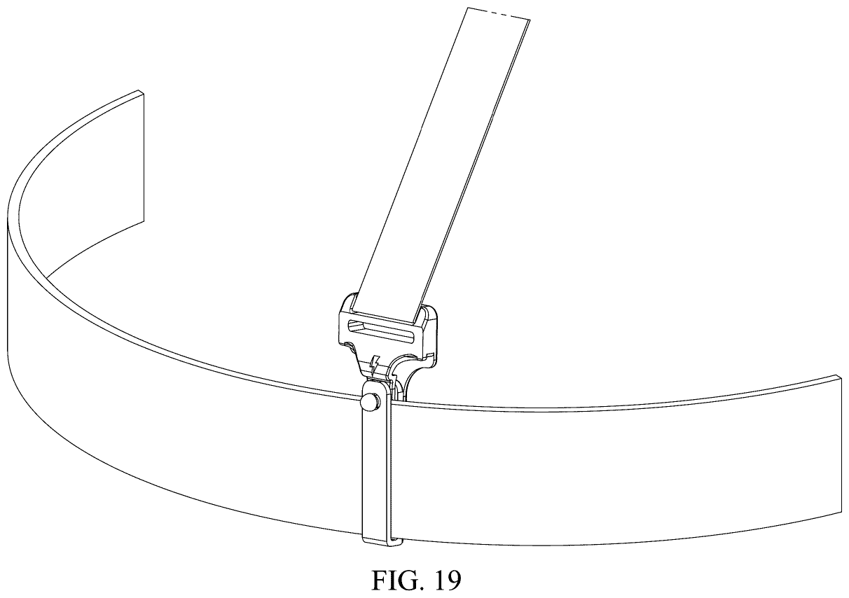

As shown in FIG. 19, the apparatus 500 is used to attach to googles. Each adapter clip component 300 is secured to a portion of googles. Specifically, the band portion of the googles is positioned within the u-shaped body 310. The band portion is located within the inside surface 312 of the adapter clip portion 300. The back surface 133 of the base portion 130 of the primary clip component 110 and the outside surface 314 of the adapter clip component 300 are located on the wearer's head with the strap 220 extending over the top of a wearer's head from ear to ear

The invention is directed to a clip device configured to attach to the headwear such as a mask or googles so that a connected strap is located over a top of the wearer's head to stabilize headwear to a wearer's head. Headwear such as a mask or goggles are secured and held in place during activities.

Further modifications and alternative embodiments of various aspects of the invention will be apparent to those skilled in the art in view of this description.

Accordingly, this description is to be construed as illustrative only and is for the purpose of teaching those skilled in the art the general manner of conducting the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. An apparatus to stabilize headwear to a wearer's head, a non-limiting, exemplary aspect of an embodiment of the invention, comprising:
a clip component connected to each end of a strap, wherein each clip component is configured to attach to the headwear so that the strap is located over a top of the wearer's head from ear to ear.

2. The apparatus of claim 1 wherein the clip component including a base portion and a flap portion moveable with respect to the base portion.

3. The apparatus of claim 2, wherein the base portion includes a first slit element and a second slit element.

4. The apparatus of claim 3, wherein the second slip element comprises a plurality of spike elements.

5. The apparatus of claim 1, wherein the strap is adjustable.

6. The apparatus of claim 2, wherein the base portion includes a protrusion element extending from a front surface of the base portion.

7. The apparatus of claim 6, wherein the protrusion element comprises a shaft portion and a bulb portion.

8. The apparatus of claim 2, wherein the flap portion is hingedly connected to the base portion.

9. The apparatus of claim 8, wherein the flap portion further comprises a slot element.

10. The apparatus of claim 2, wherein each end of the strap is routed through the first slit element and the second slit element.

11. The apparatus of claim 1, wherein the headwear is a helmet, and the clip component is secured near an ear area of the helmet.

12. The apparatus of claim 9, wherein the protrusion element is positioned through the slot element of the flap portion in a locked state.

13. The apparatus of claim 12, wherein the bulb portion pierces through both a top surface and a rear surface of the flap portion.

14. The apparatus of claim 13 wherein the bulb portion is larger in diameter than the width of the slot element so that the protrusion element fastens with an interference fit.

15. The apparatus of claim 9, wherein the slot element comprises a first section with a first opening larger than a second opening of a second section.

16. The apparatus of claim 1, wherein the clip component further comprises an adapter clip component.

17. The apparatus of claim 1, wherein the adapter clip component comprises a u-shaped body including a first leg element connected to a second leg element, the first leg element includes a first aperture element, and the second leg element includes a second aperture element.

18. The apparatus of claim 16, wherein the headwear is goggles, and the adapter clip component is secured to a band portion of the goggles.

19. A non-limiting, exemplary embodiment of a clip component, comprising:
a base portion including a first slit element and a second slit element, the second slip element comprises a plurality of spike elements, the base portion includes a protrusion element extending from a front surface of the base portion, the protrusion element comprises a shaft portion and a bulb portion,
a flap portion hingedly connected to the base portion and the flap portion moveable with respect to the base portion, the flap portion further comprises a slot element,
the protrusion element is positioned through the slot element of the flap portion in a locked state, piercing through both a top surface and a rear surface of the flap portion, and the bulb portion is larger in diameter than the width of the slot element so that the protrusion element fastens with an interference fit.

20. The clip component of claim 19 further comprising:
an adapter clip component comprises a u-shaped body including a first leg element connected to a second leg element, the first leg element includes a first aperture element, and the second leg element includes a second aperture element,
the protrusion element is positioned through the first aperture element and the second aperture element, wherein the bulb portion pierces through the body of the adapter clip component.
